(19) United States
(12) Patent Application Publication
(10) Pub. No.: US 2026/0305974 A1
(43) Pub. Date: Oct. 8, 2026

(54) APPARATUS AND METHOD FOR DETECTING INFECTION AND METABOLIC DYS-REGULATION USING BREATH

(71) Applicant: Isomark Health, Inc., Fitchburg, WI (US)

(72) Inventors: Fariba Assadi-Porter, Fitchburg, WI (US); Warren Porter, Fitchburg, WI (US); Davud Porter, Fitchburg, WI (US); Reid Formo, Blue Mounds, WI (US); Daniel Butz, McFarland, WI (US); Jack Heinemann, Waunakee, WI (US); Hesamaddin Torabi Dashti, Cambridge, MA (US)

(73) Assignee: Isomark Health, Inc., Fitchburg, WI (US)

(21) Appl. No.: 19/484,963

(22) PCT Filed: May 16, 2024
(86) PCT No.: PCT/US2024/029743

§ 371 Date: Nov. 17, 2025

Related U.S. Application Data
(60) Provisional application No. 63/502,908, filed on May 17, 2023.

(51) Int. Cl. A61B 5/08; A61B 5/00; A61B 5/097; G16H 50/20

(52) U.S. Cl. CPC A61B 5/082; A61B 5/0075; A61B 5/097; G16H 50/20; A61B 2503/40

(57) ABSTRACT

A device for predicting illness in an animal includes an outer housing that has an RFID reader attached to the housing. Also connected to the housing is a breath sample collection mask for taking a breach sample from an animal. The collection mask includes a CO₂ concentration sensor. The housing includes a spectrometer that measures the ratio of ¹²C to ¹³C in an amount of carbon dioxide present in the breath sample taken from the animal. Tubing provides an airflow connection for the breath sample from the breach sample collection mask to the spectrometer. One or more vacuum pumps in the housing pull the breath sample into the breath sample collection mask and transport it through the tubing to the analyzer. A processor in the housing includes software to analyze the collected breath sample.

### CROSS-REFERENCE TO RELATED APPLICATIONS

This application claims priority to provisional application No. 63/502,908, entitled Apparatus and Method for Detecting Bacterial and Viral Infections Based on Breath, filed May 17, 2023, the disclosure of which is hereby incorporated by reference in its entirety.

### BACKGROUND OF THE INVENTION

Maintaining animal health and metabolism is essential to the livelihood and productivity of livestock farms. However, because the livestock are often raised in large groups, with the periodic introduction of new animals, it can be difficult to control the spread of infections. When a sick animal is exposed to other animals, the contagious illness can spread quickly, resulting in high use of medications such as antibiotics, even among animals showing no sign of illness, in an attempt to prevent further spread. In some cases, when the spread of illness cannot be maintained, the resulting level of illness can be devastating to the livestock producer and the animals.

One method of reducing the spread of infection is to isolate new animals before introducing them to the general herd. However, the incubation period of some illnesses can be long. Thus, while isolation can be helpful, isolation periods can be prolonged and require significant space, which increase operational expenses. Furthermore, some animals may display little or no symptoms despite being ill and could therefore be removed from isolation and placed with the herd, thereby unknowingly creating a source of infection for herd animals.

Illnesses can also arise without the introduction of new animals. For example, the illness may spread to one or more members of the existing animal population through other pathogenic vectors such as contact with wild animals or birds, neighboring herds, or from airborne, water borne, and/or food borne pathogens, and/or by contact with farm or animal health workers. In such cases, isolation of ill animals could prevent spread to the general population but should be done as quickly as possible. Once an animal is showing symptoms, it may already have spread the illness to other animals and may be difficult or impossible to contain.

A common contagious illness among cattle is Bovine Respiratory Disease (“BRD”), also known as shipping fever. BRD is a leading cause of economic loss of feedlots, causing 75% of cattle illness and of those leading to 70% of cattle death. Bovine Respiratory Disease results in one billion dollars of loss in the United States feedlot industry annually. Because of this, workers in the cattle industry take great efforts to both prevent the disease and control the spread of the disease by isolating new and sick animals and by conducting physical examinations of animals. Even with these measures, the disease continues to be devastating when it strikes.

Methods and systems for early detection of illness, including but not limited to BRD are therefore needed to detect infections in animals and humans as early as possible in order to prevent the spread of disease.

### BRIEF SUMMARY OF THE INVENTION

In some embodiments of the present disclosure a device for predicting illness in an animal is provided. The device may include a breath sample collection mask and an analyzer configured to measure the amount or ratio of ¹²C and ¹³C in the carbon dioxide of a sample of breath from an animal, and tubing that provides an airflow connection for the breath sample from the breath sample collection mask to the analyzer. In some embodiments there is a carbon dioxide sensor in the breath sample collection mask. In some embodiments, a vacuum pump may be included that is configured to pull the breath sample into the breath sample collection mask. In some embodiments, the device also includes a processor. A source of gas in airflow connection with the tubing configured to dilute the breath sample prior to delivery to the analyzer may also be included in the device, according to some embodiments. The processor included in some embodiments, may include programming to receive a carbon dioxide concentration measurement for the breath sample that determines the amount of diluting gas needed to dilute the gas to a predetermined carbon dioxide concentration, and direct delivery of the determined amount of gas to the breath sample. In some embodiments, the processor includes programming to receive one or more biomarker measurements for the breath sample and the processor further calculates a likelihood of illness. In some embodiments, the one or more biomarker measurements comprises an amount or ratio of ¹²C and ¹³C in the carbon dioxide of the breath sample. In some embodiments, an indicator light is visible to a user, wherein the indicator light displays two different light colors to indicate health or illness of the animal based on the calculated likelihood of illness. In some embodiments, the device includes two or more breath collection pathways.

In some embodiments, a device for predicting illness in an animal includes an outer housing; an RFID reader connected to the outside of the housing; a breath sample collection mask; a CO₂ concentration sensor in the collection mask; a spectrometer in the housing, the spectrometer configured to measure the amount or ratio of ¹²C and ¹³C in the carbon dioxide of a sample of breath from an animal; tubing providing an airflow connection for the breath sample from the breath sample collection mask to the spectrometer; and one or more vacuum pumps in the housing to pull the breath sample into the breath sample collection mask and to transport it through the tubing to the analyzer. The device also includes a processor in the housing including software to control the operations of the device. In some embodiments, the device includes a wireless communication transmitter and receiver. The device may also include a display screen. The device may also include wheels attached to the bottom of the housing. In some cases, the device may include a gas line in air flow connection to the tubing configured to dilute the breath sample A gas tank may also be included in some embodiments, where the gas tank includes gas comprising oxygen and nitrogen and no carbon dioxide. The device may also include an indicator.

In some embodiments, a method of predicting illness in an animal includes: collecting a breath sample from an animal; diluting the breath sample with a gas which contains no carbon dioxide; measuring a concentration or ratio of ¹²C and ¹³C in the carbon dioxide of the breath sample; comparing the concentration or ratio of ¹²C and ¹³C in the carbon dioxide of the breath sample to a threshold value, the threshold value determined by statistical analysis of breath samples from a matched cohort of animals; and providing a prediction of health or illness of the animal based on the comparison. In some embodiments, the method may also include measuring a concentration of methane in the breath sample and comparing the concentration of methane in the breath sample to a threshold value, the threshold value determined by statistical analysis of breath sample from the matched cohort of animals and providing a prediction of health or illness based on both comparisons. In some embodiments, the statistical analysis comprises machine learning.

### BRIEF DESCRIPTION OF THE DRAWINGS

While the specification concludes with claims particularly pointing out and distinctly claiming the subject matter that is regarded as forming the various embodiments of the present disclosure, it is believed that the disclosure will be better understood from the following description taken in conjunction with the accompanying Figures, in which:

FIG. 1 a flow chart of a method of the present disclosure for collecting and analyzing a breath sample from an animal and providing a health prediction;

FIG. 2 is a perspective side view of a device of the present disclosure for collecting and analyzing a breath sample from an animal and providing a health prediction;

FIG. 3 is an example of some of the components of the device in the breath sample air flow pathway, according to various embodiments of the present disclosure;

FIG. 4 is photograph of an example of a mask for collecting a breath sample according to some embodiments of the present disclosure;

FIG. 5 shows an example of a web-based application dashboard that records and tracks animals after their breath is scanned and entered automatically into the web-based application access point for the user, according to some embodiments of the present disclosure;

FIG. 6 is an example of a distribution of values for a biomarker for a feedlot as provided by some embodiments of systems and methods of the present disclosure;

FIG. 7 is an example of an algorithm for sorting animals using the biomarker of FIG. 6, according to some embodiments of the present disclosure;

FIG. 8 is an example plot of values for two biomarkers which may be used together to sort animals, according to some embodiments of the present disclosure; and

FIG. 9 is a receiver operating characteristic (ROC) curve for the output results from the example described herein.

### DETAILED DESCRIPTION OF THE INVENTION

The following detailed description is exemplary in nature and is not intended to limit the scope, applicability, or configuration of embodiments of the present disclosure. Rather, the following description provides practical illustrations for implementing various exemplary embodiments. Utilizing the teachings provided herein, those skilled in the art may recognize that many of the examples have suitable alternatives.

The present disclosure relates to technology, systems, and methods for detecting the health of an animal, including presymptomatic risk assessment. More specifically, the present disclosure relates to apparatuses and methods for receiving a breath of an animal, analyzing the received breath, and determining whether the animal has an infection such as a bacterial, viral or fungal infection, even before symptoms become present. It will be appreciated that the use of the term “animal” herein, includes human beings, birds, and any other living specimen that may benefit from the detection of disease.

The methods and systems described herein according to some embodiments may be used to predict the likelihood of infection and detect early infection in animals such as, but not limited to livestock animals, for example, but not limited to cattle, dairy cows, calves, pigs, goats, sheep, chickens, turkeys, etc. It may also be used in other non-livestock farm animals such as horses, for example. It may also be used for domestic animals such as dogs, cats, birds, rabbits, etc. It may also be used for humans. In some embodiments, systems and methods of the present disclosure may be used on any endothermic (warm blooded) or ectothermic (cold blooded) animal.

As provided above, systems and methods of the present disclosure may be used with any warm-or cold-blooded animal. It will be recognized that while some embodiments may be discussed with regard to a specific animal, type of animal, and/or specific disease, the embodiments so described are not limited to use in those animals, types of animals, or specific disease types.

The systems and methods of the present disclosure in some embodiments may be used for periodic screening of livestock including those which appear to be healthy. It may also be used to screen new animals before introduction to a herd, or before reintroduction after a separation from the herd. In other cases, it may be used to screen animals before introduction to a group setting with other animals, such as a show, a fair, a daycare or boarding facility, or when transporting an animal from one place to another.

The systems and methods of the present disclosure according to some embodiments include the capture and analysis of exhaled breath to predict a risk of illness in an animal. The analysis may include measurement of one or more levels of exhaled gases and metabolites including, but not limited to carbon dioxide (CO₂), oxygen (O₂), methane (CH₄), ammonia (NH₃), water (H₂O), H2S and other metabolites or gases. It may also include, for example, detection of levels of ¹⁵N, ¹⁴N, ¹⁸O, ¹⁷O, ¹⁶O, ¹³C and ¹²C in the CO₂ and/or the ratios of isotopes of ¹⁵N¹⁴N in NH³, ¹⁸O/¹⁶O in O₂, ¹³C and ¹²C to each other in the exhaled CO₂.

The systems and methods may combine the analysis of the exhaled breath of an animal with other information related to that animal in order to more accurately predict a risk of illness in the animal. Other information that may be considered includes but is not limited to one or more of the animal's age, gender, and weight, genetics, and/or geographical backgrounds. Further information which may be considered includes, but is not limited to animal breed, diet, the climate where the animal resides, the time of year at testing, temperature, size of animal, astrocycle, its pregnancy status, milking cycles, and/or its fertility.

The systems and methods may analyze multiple variables together to assess the risk of infection in some embodiments. For example, in some embodiments, the system may measure the ratio of ¹³CO₂ to ¹²CO₂, and combine this with the animal's gender, age and weight, in a four-way matrix to calculate a risk of infection for a particular animal.

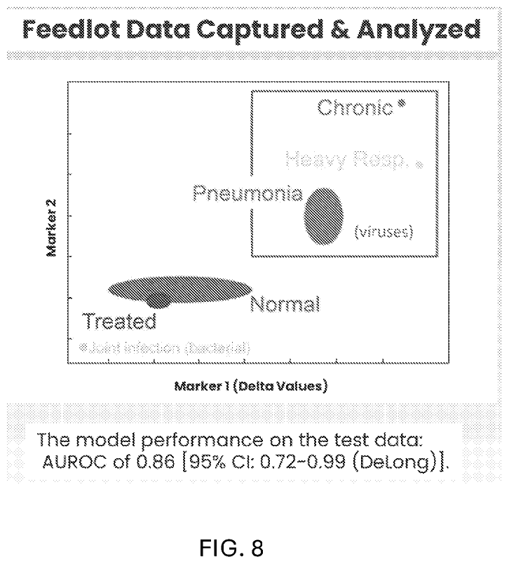

The ratio of ¹³CO₂ to ¹²CO₂ may be used in various embodiments as a reliable and highly sensitive indicator for the onset of or progression toward infection. While not intending to be bound by any theory, it is believed that the different metabolism of ¹³C versus ¹²C during infection can be explained as follows: during the early onset of proinflammatory to acute phase response, the ratio is inversely related to the severity of a developing infection. Tumor necrosis factor alpha (TNF alpha) and interleukin-1 (IL-1) may induce changes in secondary metabolism. Amino acids may rapidly be released from skeletal muscles where they can be used to make acute phase proteins or be metabolized in full. Amino acids released during the acute phase may fractionate based on the molecular weight of the amino acid. The fractionation follows the principles of the kinetic isotope effect. Approximately 1% of the world's stable carbon has the atomic weight of 13 (¹³C) and most of the remaining carbon is lighter carbon 12 (¹²C). Amino acids and products with higher amounts of ¹³C are less likely to be fully metabolized to CO₂ than those amino acids that have a majority of ¹²C, the lighter isotope. The “heavier” carbon amino acids remain as products of the acute phase (i.e., acute phase proteins) while the “lighter” carbon amino acids are more likely to be completely oxidized to CO₂. The result of this phenomenon is that the ratio of ¹³CO₂ to ¹²CO₂ in breath decreases during the infection onset. However, as the infection progresses, metabolism shifts become more anaerobic, and macronutrient metabolism changes from primarily a mixture of carbohydrates and lipids to primarily carbohydrate and body proteins. Due to isotopic discrimination against ¹³C during several steps in synthesis, lipids are 3.5% lighter than carbohydrates or proteins. Thus, a shift in macronutrient oxidation during the progression of infection causes a rapid increase in the ¹³CO₂/¹²CO₂ ratio. Because of the competing isotopic mechanisms during an untreated infection, the ratio has a higher variance than normal. In practical terms, a shift of greater than 1% above or below the baseline level may indicate the presence of infection. The amplitude and signs of change in the ¹³CO2/¹²CO2 ratios and its variance can be related to types and severity of diseases, as is shown in FIG. 8, for example. The information from the ratio may be combined with other biomarkers and individual data points with a machine learning approach to predict health status in some embodiments of the present disclosure.

Various embodiments may be used to detect illnesses and/or a risk of illness including infections such as bacterial, viral, and/or fungal infection. For example, bacterial infections which may be detected include, but are not limited to mastitis. Viral infections which may be detected include but are not limited to pneumonia. Fungal infections which may be detected include but are not limited to histoplasmosis. Some embodiments may be used to detect infection or to provide a risk of a respiratory infection including infections of the lung and/or respiratory tract including for example, pneumonia, influenza, and/or Bovine Respiratory Disease, for example.

The analysis of exhaled breath, alone or in combination with other animal specific variables, may be used to predict a risk of infection prior to the presence of any physical symptoms in some embodiments of the present disclosure. For example, illness may be detected as early as 4-6 hours after the introduction of the disease organism in some embodiments. In some cases, the systems and methods may predict a risk of infection generally, while in other cases they may predict a risk or severity of a specific infection or specific type of infection.

Early identification of an increased risk of infection may reduce the spread of illness to other animals through earlier isolation of the at-risk animal, earlier medical intervention such as antibiotic administration to the at-risk animal, and/or other animals in close contact with the at-risk animal.

The systems and methods of the present disclosure according to some embodiments can provide rapid analysis of the breath, quickly producing a risk assessment. For example, the results may be provided in less than one minute in some embodiments, such as about 5 to about 59 seconds, or less than 40 seconds, such as about 3 to about 39 seconds. In this way, the risk assessment results can be provided to the animal worker while the worker is still working with that animal and the animal is still in a head restraint. If the results indicate an increased risk of infection, further steps can be taken prior to releasing the animal from the head restraint, such as diverting the animal into isolation and/or delivery of a medication or vaccination.

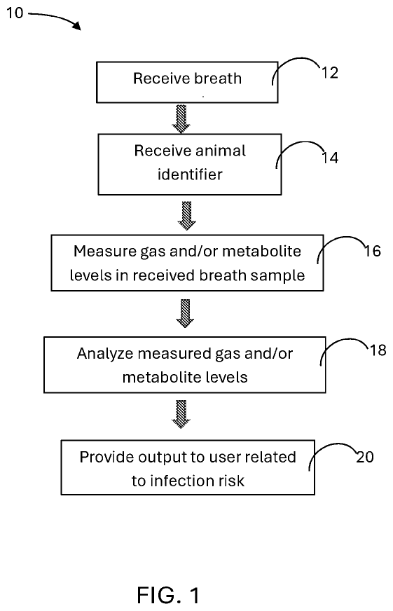

An example of a method of illness risk assessment using an illness risk assessment system is shown in FIG. 1. In this example, method 10 begins with collection of a breath sample from an animal using the system and receipt of the sample in step 12. In step 14, a user inputs an identifier of the individual and the system receives the input user identifier. For example, the user may scan a unique RFID tag or other identifier using a scanner included in the system. Alternatively, step 14 could occur prior to step 12. If the animal is already included in the system database, other information about the animal may already be known. However, if the animal is new to the system, the user may also input other information about the animal such as gender, age, weight at this time after inputting the identifier.

In step 16, after receiving an adequate breath sample, the system measures analytes in the breath. For example, the system may detect and determine the quantity and/or ratio of the analyte or analytes. The measurement may include gas and/or metabolite levels and/or ratios, such as CO₂, O₂, methane, ethane, ammonia, water, and/or ¹³C to ¹²C (or vice versa) ratio in CO₂.

The measured values obtained from the breath sample may then be analyzed in step 18, during which one or more measurements, optionally in combination with one or more other individual parameters (such as age, gender, weight). This analysis may be performed using an algorithm to predict a risk of infection, which is discussed in detail further below.

In step 20, the system provides an output to a user related to the risk of infection predicted in step 18. This output may be immediate feedback (such as in less than 1 minute or less than 30 seconds for example) provided on the breath collection and analysis system, or may be provided at a later time. The output may be a simple binary output, such as sick or not sick, based on whether the risk of infection determined in step 18 falls above or below a threshold. The output may optionally include a third category, directing the user to retest, if the result is inconclusive or potentially flawed. In such systems, the output may be provided on a user interface such as a screen, or through a light or sound. For example, colored lights may be used to indicate the risk of infection, such as red for sick, green for not sick and a third color such as white for retesting. Other output may be provided instead or in addition, such as an auditory signal or written words or pictures. For example, FIG. 5 shows an example of outputs from a series of measurements on several animals, one of which is sick (shown in red) and the rest which are health (shown in green).

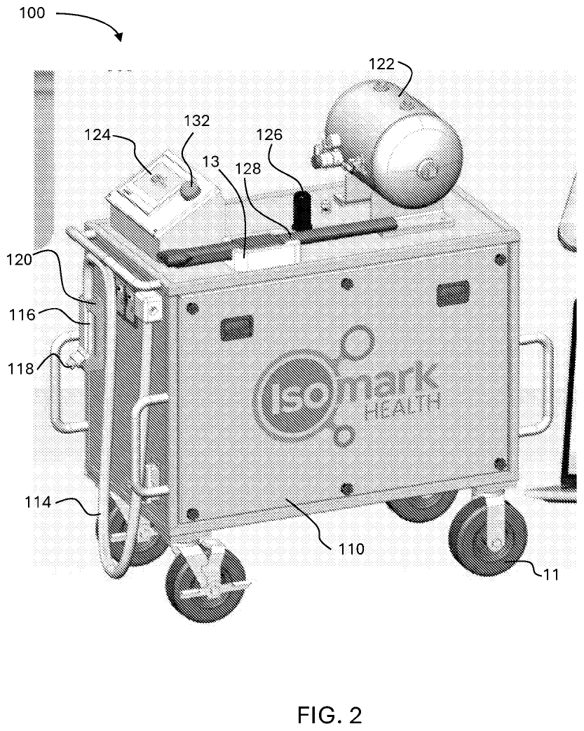

An example of a device which may be used to gather and analyze the breath of an animal according to embodiments of the present disclosure is shown in FIG. 2. The device 100 includes a housing 110 which may optionally include wheels 112 to make the device 100 mobile. Flexible tubing 114 at a front end of the system includes a first end connected to and extending from the housing 110 and a second end to which a mask (not shown) may be attached. The second end of the tubing may terminate at a rigid handle 116 which may form a connector 118 at its distal end, configured to securely connect to the mask. In the embodiment shown, the handle 116 is supported by a handle holder 120 on the front end of the device, which keeps the mask off the ground and easy to find.

The device 100 may also include an optional gas tank 122 which provides gas used during the analysis of the breath sample, referred to herein as zero gas, which will be described further later in this disclosure. However, the zero gas may alternatively be provided by a separate gas tank which is not a part of the device 100, such as in systems which are not mobile, or which are mobile but are moved only short distances still within useful reach of the stationary gas tank. The gas tank may include one or more regulators to modulate the flow of gas.

The device may include a processor, not shown, which may include software which controls the functioning of the device 100. The device 100 may further include a user interface 124 which may be a screen such as a touch screen to display information and instructions for the user, for example, and optionally to receive user input, and may connect with the processor.

The device 100 may communicate data through hard wired and/or wireless connection. In the example, the device may include a Wi-Fi transmitter and receiver 126 for sending and receiving data, such as from the cloud. Results may be transmitted via wired or wireless connection to a cloud-based system which may electronically integrate the results with the animal's medical records and make the results electronically available to a user at a later time.

The device 100 may include a reader 128 which scans or reads a unique identifier on the animal such as an RFID tag. In this example, the reader 128 is an electronic ID (EID) reader. A user uses the reader to scan a unique identifier on the animal, such as an RFID ear tag. The device may also include a reader holder 130 which holds the reader 128 for ease of locating it when needed. Other types of readers may alternatively be used. Alternatively, the unique identifier such as the alphanumeric code or name could be manually entered. The scanned identification may be automatically provided to the processor and associated with the collected breath sample results. In alternative embodiments, the reader may be incorporated into the handle 116 so that both the scanning of the unique identifier and the collection of breath may be performed using the same handheld component.

The device 100 may further include an indicator light 132 which may be illuminated to display and convey the result of the analysis to the user. The indicator light 132 may remain dark until the result is ready and may illuminate with different colors to indicate the result. For example, a green light as shown may indicate the animal's results are consistent with a healthy animal, with the risk of infection falling below a threshold. A red light may indicate that the animal's results are consistent with an elevated risk of infection above the threshold. A third color, such as white, may indicate that the animal measurement should be repeated. In this way, the device 100 may give nearly immediate feedback to the user who is still working with the animal and before proceeding to test the next animal. While this embodiment is described using specific colors to indicate a particular test status, it will be understood that any color and/or other symbol or method of displaying test results could be used.

The device may further include various internal components, not shown in FIG. 2, which may function in the breath sample collection and analysis process. For example, the device may include one or more sensors such as CO₂ concentration sensors, and one or more analyzers, such as infrared spectrometers, mass spectrometers or cavity ring down laser spectrometers, and/or any other analysis equipment that may measure atomic masses, and/or identify a specific molecule and/or concentration from the breath, for example. There is no practical limit on the type of analysis and/or measuring device or tool that may be incorporated into embodiments of the present disclosure. One spectrometer which may be used in various embodiments is a Picarro cavity ring down laser spectrometer. The spectrometer may be configured to measure the quantity or relative quantities of ¹³C and ¹²C in the CO₂ of the sample through cavity spectroscopy, for example. In some embodiments, the one or more analyzers may include a spectrometer that measures a ratio of ¹³C to ¹²C (or vice versa) in CO₂, methane, and/or other molecules in the breath sample that may be diagnostic.

For measuring a ratio of ¹³C to ¹²C (or vice versa) in CO₂, the breath sample may be combined with zero air to adjust the amount of CO₂ present in the breath sample. The zero air may be gas or air which may include all of the components of air except for CO₂, or which may include only the primary components of air, oxygen and nitrogen, but no CO₂. The amount of zero air to be added to each breath sample may vary among individuals. Therefore, a CO₂ sensor may detect the baseline amount of CO₂ present in the sample, and zero air may be used to dilute the level of CO₂ to achieve the predetermined value used for uniform testing, to equalize sample results among individuals. By diluting the quantity of CO₂ to bring it to a uniform value using zero air, the analyzer may more accurately detect the ratio of ¹³C to ¹²C in the CO₂. As such, various embodiments may include a CO₂ sensor to detect CO₂ levels in a breath sample. The device 100 may then calculate the amount of zero air needed and may then deliver that amount to the breath sample. The CO₂ may then be measured after the dilution to verify concentration. The device may then deliver the diluted breath sample to the sample chamber of the analyzer.

The device 100 may include one or more CO₂ sensors to detect the adequacy of a breath sample. For example, CO₂ sensors may be located in the breath collection area of a collection mask and in a sample reservoir within the device, for example. A CO₂ sensor in the collection mask may detect the concentration of CO₂ collecting in the mask to determine when an adequate breath sample has been collected, such as when the CO₂ ppm is above a predetermined threshold value, which may be between about 15 and about 20 times greater than the atmospheric CO₂. For example, when the device 100 is used to collect breath samples from cattle, the device threshold value for CO₂ concentration in the mask to indicate an adequate breath sample may be about 10,000 ppm (which in some embodiments may be a minimum level for an adequate breach sample), though other values may alternatively be used.

The device 100 may further include a rechargeable battery to provide power to the systems, even when away from a source of electricity, as well as a power cord for connection to an external source of electrical power. The external power may be used to operate the device and to recharge the battery. Alternatively, the device 100 may not include a battery and may only include a power cord for attachment to an external source of electrical power, such as for applications which are not mobile.

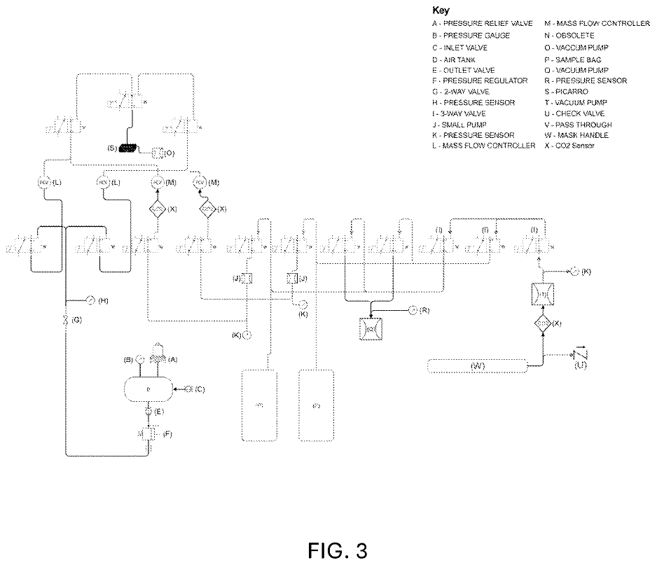

The device 100 may further include a plurality of valves, sensors and/or controllers to regulate and control the flow of breath and gases through the device. The device 100 may further include one or more vacuum pumps or other pumps to pull the breath sample from the mask, through the flexible tubing 110, through internal system tubing, and to a sample chamber for analysis. An example of the configuration of the components of the device 100 is shown in the schematic shown in FIG. 3, which will be described further below.

The device 100 further includes the computer and electronic elements needed to operate the system including, for example, a motherboard, one or more processors such as a CPU, memory, storage, a graphical user interface, tangible or non-transitory computer readable or computer accessible media, and software programing instructions or scripts to control the operations of the device 100 as well as the data analysis and algorithms used to predict the health status of the animals. The computer elements may be in electronic communication, such as remote communication, with servers which may also store some of the data collected by the device and perform some or all of the algorithms.

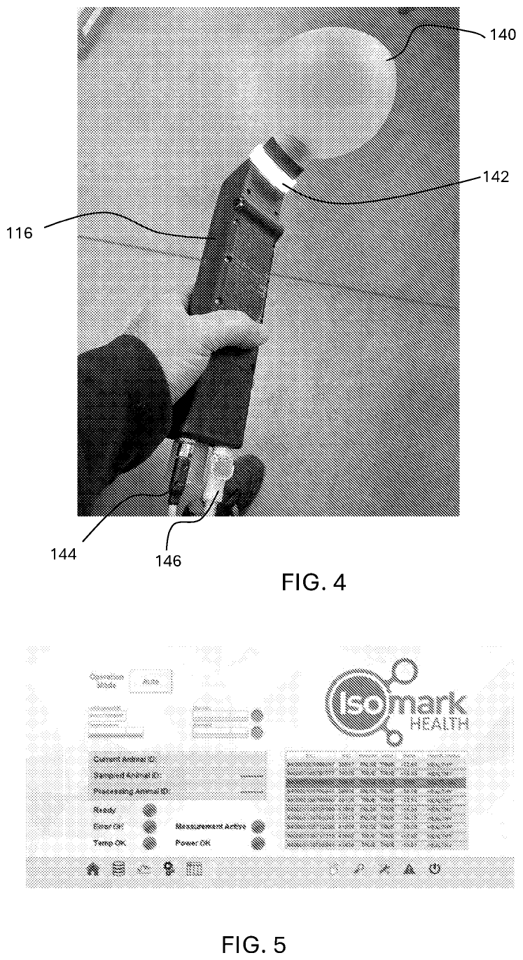

An example of a collection mask 140 connected to a distal end of a handle 116 according to some embodiments of the present disclosure is shown in FIG. 4. The collection mask may generally be made of a flexible material such as rubber or silicon and may be approximately conical or bowl shaped to comfortably fit over the nose and mouth of an animal. Various embodiments are able to collect a sample from only one or two respiratory cycles of the animal, in less than 5 seconds, or less than 4 seconds, or less than 3 seconds, such as about 2.5 seconds, or sample collection time with the collection mask 140 over the animal's nose and mouth. It will be appreciated that any other suitable material or combination of materials may be used for the mask, and/or the mask may have any suitable configuration for breach collection from a given animal.

The handle 116 may also include an indicator light 142 to indicate the status of the device 100 with regard to breath capture. For example, the indicator light may display first a color or signal to indicate that the device 100 is ready to capture and receive a breath sample. It may display a second color or signal to indicate that an adequate sample has been captured. It may display a third color or signal to indicate that the device 100 is processing the sample. For example, the signal may be a pattern such as flashing, or a steady display of single color or alternating colors. However, a different light signal may be used for each of the three statuses, and other status may also be identified using a fourth color or signal, etc. Additionally, the signal may be an auditory signal, or any other signal to indicate status.

By locating the indicator light 142 on the handle 116, and particularly at the distal end of the handle 116 between a user's hand when using the handle and the collection mask 140, it can be easily seen by the user while facing and working with the animal from whom the user is collecting the sample. The user does not need to turn around, or reposition the device, in order to see the indicator light 142 and observe the change of the light or signal. Rather, the user can see the change of the light or signal immediately, such as while looking at and holding the collection mask 140 over the animal's face. In this way, the process of using the collection mask is quick and easy, with no time wasted due to uncertainty regarding the adequacy of a breath sample. There may be a button or other switch on the handle in some embodiments that allows the operator to rapidly activate breach collection by triggering the vacuum that pulls breach into the mask. The CO₂ sensor can determine if the minimum breach of around 10,000 ppm CO₂ has been collected. During this determination a blinking light may indicate breach collection is in progress. Once enough CO₂ in the breath has been captured the light color may change to, for example, a spinning color to indicate that the sample is being analyzed. Of course, other indicator signals may be used to indicate different sample states.

In some embodiments, device 100 may enable more efficient sampling of animals by capturing breath samples from animals before the device 100 completes the analysis of a breath sample from a previously tested animal. In this way, there may be little or no wait time between animals, as the sample collection process can continue during analysis. For example, the device may have a single breath collection mask, but the collected breath may be transported to one or two or more separate and identical sample reservoirs prior to analysis. The breath sample may be held in the sample reservoir until the single analyzer is ready to test the sample, such as until testing of a previous sample has been completed. The breath sample may then be transported into the analyzer to complete analysis of the breath sample.

A detailed example of a device including two pathways or channels for breath sampling is shown in FIG. 3, according to some embodiments of the present disclosure. The breath sample may be pulled into the collection mask by a vacuum pump, in some embodiments. When the CO₂ sensor in the collection mask handle detects an adequate breath sample, a vacuum pump may pull the breath sample through the mask handle into one of two sample reservoirs, also referred to as sample bags, which hold the samples, as directed by the valves. When the spectrometer is ready to test the next sample, the breath sample may be transported out of the reservoir and the CO₂ level may be measured again between the reservoir and the analyzer, to determine the CO₂ level in the sample and the amount of zero air required to properly dilute the CO₂. The appropriate amount of zero air may then be delivered to the breath sample to dilute the breath sample through a mass flow controller. In this example, a single zero air tank may be used, and the supply of zero air may be split into two pathways to deliver air to the two breath sample pathways. The diluted breath sample may then be delivered to the single analyzer for breath analysis. In alternative embodiments, the device may include only a single breath collection reservoir and pathway, or more than two, such as three or four, for example.

An algorithm may be used to determine whether an animal is presymptomatic and at increased risk showing signs of illness within a time period such as 14 days or 30 days. This algorithm may be developed using a training set of animals of the same type as those who will be tested using the algorithm. For example, the algorithm may be developed using machine learning and/or neural networks. Machine learning methods which may be used to develop the algorithm may include one or more of supervised learning (such as regression algorithms, classification algorithms, Naïve Bayes classifiers, neural networks, and random forest algorithms), unsupervised learning (such as K-means clustering, hierarchical clustering, and probabilistic clustering), self-supervised learning, semi-supervised learning and/or reinforcement learning, for example.

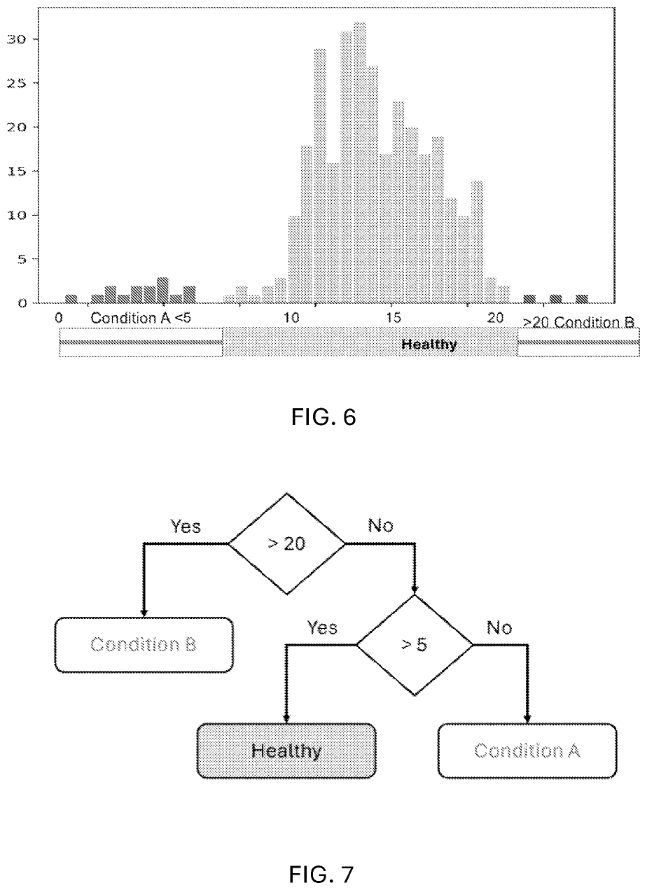

The algorithm may include cut-off values for particular biomarkers. For example, as shown in the graph in FIG. 6 and the flowchart in FIG. 7, a biomarker may have a healthy middle range, while values both above and below the normal range indicate an increased likelihood of illness. In this example, the cut off values are 5 and 20, with a value of less than 5 indicating an increased risk of condition A, and a value of greater than 20 indicating an increased risk of condition B.

An example of how an algorithm including two biomarkers may be used to screen animals for illness is shown in the grid presented in FIG. 8. In this example, the combination of two biomarkers may be used to predict the existence of a plurality of illnesses. In this example, the cutoff values for each biomarker depend not only upon that biomarker but also the other biomarker. A normal value falls within a range of values for both biomarker 1 and biomarker 2. Abnormal values can be used to predict illnesses, based on the combination of the two biomarkers.

A similar combination of biomarkers and other animal specific data may be used in multiple dimensions to improve the accuracy of prediction. The development of an algorithm including multiple biomarkers and/or other data points is highly complex and may rely upon machine learning to be developed.

The severity of a disease condition using a small set of biomarkers may require using advanced analytical algorithm in some embodiments, for example supervised max-margin kernel machine (support vector machines) that use a training dataset that may identify a hyperplane that separates healthy from sick animals.

### EXAMPLE

As shown in FIG. 8, different disease states may have different interactions between two or more biomarkers. In FIG. 8, a few conditions such as chronic infections, pneumonia and heavy respiratory may be different than in the healthy condition range and bacterially infected joint infection. The disease complexity can be multidimensional and may include interaction between more than two biomarkers. In the example shown, the interactions are for ¹³C/¹²C ratios (X-axis) v. methane (Y-axis) and the ranges for the different disease types are shown in circles or oblongs.

Breath samples were collected from cattle in feedlots across the United States and analyzed according to embodiments of the present disclosure. About 20,000 breath samples were collected. The breath samples were obtained as cattle entered the feedlot. Electronic identification (EID) numbers were individually assigned. The EID tag of each animal was then used to initiate the sample collection process, using the collection mask of the device described in this disclosure. The device analyzed the breath samples of each animal including ¹³CO₂/¹²CO₂ and other biomarkers within 30 seconds.

Health data was obtained from the feedlots' health management software. Focusing on the first 14 days, health events were noted in 11.1% of the animals measured, of which 49.3% were lung related infections. The system and methods of the present disclosure was able to distinguish a variety of disease conditions including acute and chronic lung conditions of bacterial or viral origins.

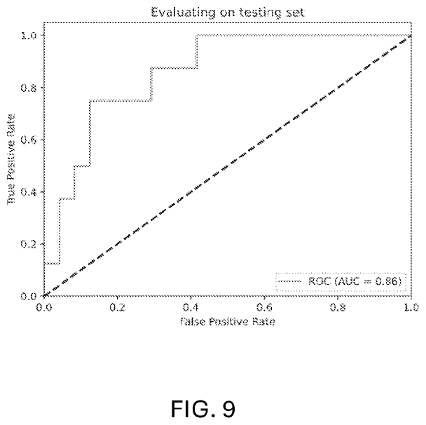

To train the prediction algorithm, 80% of the healthy and unhealthy animals were selected at random to construct a derivation dataset, and the remaining 20% of the data was considered as the validation dataset. The supervised machine learning algorithm created using the derivation dataset was used to predict the health status of animals in the remaining validation set. As shown in FIG. 9, the area under the curve (AUC) of the receiver operating characteristic curve was 0.86 after 5-fold cross-validation, representing a sensitivity of 78% and a specificity of 74%. Independent validation of the detection algorithm, using 11 different classical and machine learning techniques, confirmed that the predictive model predicted sickness.

As used herein, the terms “substantially” or “generally” refer to the complete or near complete extent or degree of an action, characteristic, property, state, structure, item, or result. For example, an object that is “substantially” or “generally” enclosed would mean that the object is either completely enclosed or nearly completely enclosed. The exact allowable degree of deviation from absolute completeness may in some cases depend on the specific context. However, the nearness of completion will be to have generally the same overall result as if absolute and total completion were obtained. The use of “substantially” or “generally” is equally applicable when used in a negative connotation to refer to the complete or near complete lack of an action, characteristic, property, state, structure, item, or result. For example, an element, combination, embodiment, or composition that is “substantially free of” or “generally free of” an element may still actually contain such element as long as there is no significant effect thereof.

In the foregoing description various embodiments of the invention have been presented for the purpose of illustration and description. They are not intended to be exhaustive or to limit the invention to the precise form disclosed. Obvious modifications or variations are possible in light of the above teachings. The embodiments were chosen and described to provide illustrations of the principals of the invention and its practical application, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

What is claimed is:

1. A system for predicting illness in an animal, the system comprising:
a breath sample collection mask;
an analyzer configured to measure the ratio of ¹²C to ¹³C in an amount of carbon dioxide from a sample of breath from an animal taken using the breath sample collection mask; and
tubing providing an airflow connection for the breath sample from the breath sample collection mask to the analyzer.

2. The system of claim 1 further comprising a carbon dioxide sensor in the breath sample collection mask.

3. The system of claim 2 further comprising a vacuum pump that pulls the breath sample into the breath sample collection mask.

4. The system of claim 3 further comprising a processor.

5. The system of claim 4 further comprising a source of gas in airflow connection with the tubing that dilutes the breath sample prior to delivery to the analyzer.

6. The system of claim 5 wherein the processor includes programming to receive a carbon dioxide concentration measurement for the breath sample, and then determine the amount of diluting gas needed to dilute the gas to a predetermined carbon dioxide concentration, and then direct delivery of the determined amount of gas to the breath sample.

7. The system of claim 3 wherein the processor includes programming to receive one or more biomarker measurements for the breath sample and calculate a likelihood of illness.

8. The system of claim 7 wherein the one or more biomarker measurements comprise the ratio of ¹²C to ¹³C in the carbon dioxide of the breath sample.

9. The system of claim 7 further comprising a handle with an indicator light that is visible on the handle to a user, wherein the indicator light displays two different light colors to indicate a health or illness of the animal based on a calculated likelihood of illness.

10. The system of claim 1 comprising two or more breath collection pathways.

11. A device for predicting illness in an animal, the device comprising:
an outer housing;
an RFID reader connected to an outside of the outer housing;
a breath sample collection mask connected to the outer housing;
a CO₂ concentration sensor in the collection mask;
a spectrometer in the outer housing, the spectrometer configured to measure the ratio of ¹²C to ¹³C in an amount of carbon dioxide in a sample of breath collected from an animal;
tubing providing an airflow connection for the breath sample from the breath sample collection mask to the spectrometer;
one or more vacuum pumps in the housing to pull the breath sample into the breath sample collection mask and to transport it through the tubing to the analyzer; and
a processor in the housing for analyzing the collected breath sample.

12. The device of claim 11 further comprising a wireless communication transmitter and receiver in the housing.

13. The device of claim 12 further comprising a display screen on the housing.

14. The device claim 11 further comprising wheels attached to a bottom of the housing.

15. The device of claim 11 further comprising a gas line in air flow connection to the tubing configured to dilute the breath sample.

16. The device of claim 15 further comprising a gas tank including gas comprising oxygen and nitrogen and no carbon dioxide.

17. The device of claim 11 further comprising an indicator light on the housing.

18. A method of predicting illness in an animal, the method comprising the steps of:
collecting a breath sample from an animal;
diluting the breath sample with a gas that contains no carbon dioxide;
measuring a concentration or ratio of ¹²C and ¹³C in an amount of carbon dioxide from the breath sample;
comparing the concentration or ratio of ¹²C and ¹³C in the carbon dioxide of the breath sample to a threshold value, the threshold value determined by statistical analysis of breath samples from a matched cohort of animals;
providing a prediction of health or illness of the animal based on the comparison.

19. The method of claim 18 further comprising measuring a concentration of methane in the breath sample and comparing the concentration of methane in the breath sample to a threshold value, the threshold value determined by statistical analysis of the breath sample from the matched cohort of animals and providing a prediction of health or illness based on both comparisons.

20. The method of claim 19 wherein the statistical analysis comprises machine learning.
